(19) United States
(12) Patent Application Publication
(10) Pub. No.: US 2012/0052544 A1
(43) Pub. Date: Mar. 1, 2012

(54) ENUCLEATION OF CELLS WITH PSORALENS

(76) Inventor: Thomas J. McGarry, Wilmette, IL (US)

(21) Appl. No.: 13/211,840

(22) Filed: Aug. 17, 2011

Related U.S. Application Data
(60) Provisional application No. 61/376,416, filed on Aug. 24, 2010.

(51) Int. Cl. C12N 13/00; C12M 1/42; C12N 5/075; C12N 5/071; C12N 5/076

(52) U.S. Cl. 435/173.1; 435/325; 435/283.1

(57) ABSTRACT

The present invention provides compositions and methods for enucleation of cells. In particular, the present invention provides the use of psoralens in enucleation of feeder and/or eggs cells.

### CROSS-REFERENCE TO RELATED APPLICATIONS

The present invention claims priority to U.S. Provisional Patent Application 61/376,416, filed Aug. 24, 2010, which is herein incorporated by reference in its entirety.

### FIELD OF THE INVENTION

The present invention provides compositions and methods for enucleation of cells. In particular, the present invention provides the use of psoralens in enucleation of feeder and/or eggs cells.

### BACKGROUND

In order to generate feeder layers for cultured cells or to prepare recipient egg cells for nuclear transfer the cell nucleus must be enucleated (e.g. inactivated or destroyed). Existing enucleation techniques are cumbersome and/or employ toxic chemicals.

Stem cells and other fastidious cell types are often cultured with feeder cells that provide an appropriate niche to maintain them in their natural physiological state (Thomson et al., 1998). Feeder cells may be gamma-irradiated or treated with the radiomimetic compound mitomycin C in order to prevent them from proliferating and overgrowing the culture. These agents introduce double-stranded breaks or cross-links into the nuclear DNA, thereby interfering with replication and activating checkpoint mechanisms that arrest the cell cycle. The existing techniques to prepare feeders have serious limitations. Gamma-irradiation requires an expensive cesium source that is usually available off-site and requires compliance with strict safety regulations. Mitomycin C is highly toxic and requires several hours of treatment to be effective.

Similarly, the egg cell nucleus must be removed or destroyed during somatic cell nuclear transfer experiments (Li et al., 2004). Manual enucleation does not damage mammalian eggs, but it is time consuming, requires technical expertise, and cannot be used for species that have opaque eggs (Liu et al., 2000a). A number of alternatives to manual enucleation have been developed (Gurdon, 1960; Tatham et al., 1995) (Fulka and Moor, 1993; Wang et al., 2001; Kawakami et al., 2003; Vajta et al., 2005; Li et al., 2006), but these are damaging to the eggs (Smith, 1993) and embryonic development after nuclear transfer is frequently abnormal.

### SUMMARY OF THE INVENTION

In some embodiments, the present invention provides a method of enucleating cells comprising treating cells with a psoralen compound and UV light. In some embodiments, the psoralen compound comprises a substituted psoralen compound. In some embodiments, the psoralen compound comprises aminomethyl-4, 5′, 8-trimethylpsoralen (AMT). In some embodiments, the psoralen compound comprises 8-methoxypsoralen (MOP). In some embodiments, the psoralen compound comprises an isomer or derivative of psoralen. In some embodiments, the cells are incubated with said psoralen compound for 1-10 minutes. In some embodiments, the cells are incubated with said psoralen compound for 1-10 minutes prior to exposure with UV light. In some embodiments, the UV light comprises long-wave UV light. In some embodiments, the psoralen compound and said UV light are administered concurrently. In some embodiments, the cells are exposed to said UV light for 1-10 minutes. In some embodiments, the UV light is administered subsequent to the psoralen compound. In some embodiments, the cells comprise eggs cells or sperm cells.

In some embodiments, the present invention provides a composition comprising one or more enucleated cells, wherein the cells have been enucleated by exposure to a psoralen compound and UV light. In some embodiments, the cells comprise eggs cells or sperm cells. In some embodiments, the cells comprise feeder cells. In some embodiments, the psoralen compound comprises AMT or MOP. In some embodiments, the UV light comprises long-wave UV light. In some embodiments, the cells comprise a nucleus. In some embodiments, the cells are incapable of DNA replication. In some embodiments, the cells comprise covalent cross-strand cross-links.

In some embodiments, the present invention provides systems comprising: a) a device configured to produce UV light; b) a psoralen compound; and c) a plurality of cells.

### BRIEF DESCRIPTION OF THE FIGURES

The following drawings form part of the present specification and are included to further illustrate aspects of the present invention. The drawings highlight exemplary embodiments of the present invention, but should not be viewed as limiting the scope of the invention. The invention may be better understood by reference to the drawings in combination with the detailed description of the specific embodiments presented herein.

FIG. 1 shows diagrams, images, and graphs demonstrating the enucleation of Xenopus eggs with psoralen and ultraviolet light: (A) experimental design for enucleating eggs; (B) top, appearance of typical diploid and haploid Xenopus embryos; bottom, typical chromosome spreads from diploid and haploid embryos; (C) graph depicting the results of enucleation treatment on Xenopus; (D) DNA content of primary cultured cells from control embryos, embryos derived from AMT+UV-treated eggs, and embryos derived from AMT-treated or UV-treated eggs; (E) eggs from a CMV-GFP female were left untreated or enucleated with AMT and UV light then fertilized with sperm, embryos were scored for GFP fluorescence at stage 40-45; (F) left, eggs from a CMV-GFP(+) female were enucleated with AMT and UV light and transplanted with a GFP(−) blastula cell nucleus; right, eggs from a GFP(−) female were enucleated with AMT and UV light then transplanted with a CMV-GFP(+) blastula cell nucleus.

FIG. 2 shows: (A) experimental design for enucleating sperm; (B) wild-type Xenopus sperm treated with AMT+UV light then used to fertilize eggs from an albino female; when controls reached stage 40-45, the percentage of embryos having a typical diploid, haploid, or abnormal appearance was scored; (C) typical embryos derived from the experiment described in part (B); (D) Xenopus eggs or sperm were treated with different concentrations of AMT and irradiated for 5 minutes before fertilization, the number of haploid embryos was scored; and (E) Xenopus eggs or sperm were treated with 50 μM AMT and irradiated for different times before fertilization, the number of haploid embryos was scored.

FIG. 3 shows Xenopus eggs either left untreated or enucleated with AMT+UV light, then fertilized with sperm that were either untreated or enucleated with AMT+UV light. (Top) Blastula stage embryos from a control fertilization (left) and a fertilization where both egg and sperm were treated with AMT+UV light (right). (Bottom) Percentage of normally and abnormally cleaving embryos from control fertilizations and fertilizations with enucleated eggs, enucleated sperm, or both.

FIG. 4 shows psoralen and UV activation of the DNA replication checkpoint: (A) Mouse eggs were left untreated or treated with 4-6 μM AMT and long-wave UV light for 30 seconds, they were parthogenetically activated and four days later the extent of development was scored; (B) mouse sperm were treated with 5 μM AMT, irradiated with a 100 W UV source for 15 seconds, and used to fertilize eggs in vitro, arrows indicate the two pronuclei; (C) HeLa cells were incubated in PBS containing different concentrations of MOP and irradiated for 1 min, after three days of culture the number of cells and the percent viability were determined; (D) HeLa cells were left untreated or irradiated in PBS containing 5 μM MOP for 1 min then transferred to culture medium, the number of cells and the percent viability were measured; (E) HeLa cells were incubated in PBS containing 5 μM MOP and irradiated with a 4W handheld long-wave UV source for 1 minute. At various times afterwards the amount of phosphorylated Chk1 was determined by immunoblotting (arrowhead, phospho-Ser³⁴⁵ Chk1; asterisk, cross-reacting band that serves as a loading control); (F) exponentially growing HeLa cells were treated as in (E); after 18 hours of culture the DNA content was measured by flow cytometry.

FIG. 5 shows feeder cells prepared by psoralen+UV treatment: (A) a monolayer of primary MEFs was incubated in PBS containing 5 μM AMT then irradiated for 30 sec (left) or left untreated (right), hESCs were plated on the cells and colonies were photographed after 6 days (Scale bar=200 μM); (B) AMT+UV-treated cells were stained with antibodies to Oct3/4 and with Hoechst dye (Scale bar=10 μM); (C) CIN612 keratinocyte precursors were plated on untreated J2 fibroblasts (right) or fibroblasts that had been irradiated for 2 minutes in the presence of 5 μM AMT (Scale bar=100 μM).

FIG. 6 (left) primary MEFs were either gamma-irradiated or treated with AMT+UV light then cultured for 1-8 days; at various times, hESCs were plated on them and cultured for an additional 4 days; hESC colonies were fixed with formaldehyde and scored as undifferentiated or differentiated based on the sharpness of the colony border and the intensity of Oct3/4 staining; the percentage of undifferentiated colonies is shown (Black, AMT+UV; Gray, gamma-IR). (Right) Typical appearance of differentiated (a-c; g-i) and undifferentiated (d-f; j-l) hESC colonies plated on AMT+UV (a-f) or gamma-irradiated feeders (g-1) six days after treatment (Red, Oct3/4 staining; Blue, DAPI) (Scale bar=200 μM).

### Definitions

As used herein, the term “enucleation” refers to a process by which cell division within a cell is arrested. “Enucleation” may be performed by interfering with DNA replication to such a degree that cell division is no longer possible. Although “enucleation” can be achieved by removal of the nucleus from a cell, physical removal of the nucleus or termination of all nuclear function is not necessary to achieve “enucleation.” For example an “enucleated” cell may remain transcriptionally active.

As used herein, the terms “psoralens” or “psoralen compounds” refer to any derivatives or substituted versions of the parent compound psoralen (IUPAC name: 7H-furo[3,2-g]chromen-7-one):

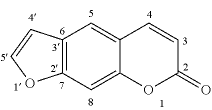

“Psoralens” or “psoralen compounds” are also referred to as “furocoumarins,” as they are structurally related to coumarin with the addition of a furan ring. The term “Psoralens” refers to psoralen derivatives (e.g. imperatorin, xanthotoxin, bergapten, nodekenetin, etc.), substituted psoralen (aminomethyl-4,5′,8-trimethylpsoralen (AMT); 8-methoxypsoralen (MOP); etc.), isomers of psoralen (e.g. angelicin), and combinations thereof (e.g. substituted derivatives of psoralen isomers).

### DETAILED DESCRIPTION OF THE INVENTION

The present invention provides compositions, systems, and methods for enucleation of cells. In some embodiments, the present invention provides enucleation of cells through treatment with polycyclic compounds (e.g. polyaromatic compounds (e.g. furocumarin derivatives))) and exposure to light (e.g. ultraviolet (UV) light (e.g. long-wave UV light). In particular, the present invention provides psoralen compounds and long-wave UV light in enucleation of feeder and/or eggs cells.

In some embodiments, the present invention provides methods to generate feeder layers and enucleated eggs by treating cells with psoralens and ultraviolet light. Although the present invention is not limited to any particular mechanism of action and an understanding of the mechanism of action is not necessary to practice the present invention, psoralens are tricyclic polyaromatic furocoumarin derivatives that intercalate between the base pairs of double-stranded DNA molecules (Cimino et al., 1985). Psoralens form covalent adducts with thymidine residues when irradiated with long-wave UV light (e.g. 300-400 nm). This reaction introduces cross-links between the two DNA strands at d(TpA) sites. Unlike mitomycin C, a compound commonly used in enucleation of cells, psoralens are non-toxic and are commonly taken internally by human subjects (Stern, 2007).

In some embodiments, the present invention provides administering psoralen compounds and UV light to cells to prevent nuclear replication. In some embodiments, the present invention provides psoralen compounds for the enucleation of cells. In some embodiments, psoralen compounds comprise any compounds derived from psoralen. In some embodiments, psoralen compounds comprise any isomers of psoralen. In some embodiments, psoralen compounds comprise substituted psoralen compounds. In some embodiments, psoralen compounds comprise compounds derived from psoralen displaying one or more substituents or functional groups. In some embodiments, psoralen compounds comprise psoralen isomers displaying one or more substituents or functional groups.

In some embodiments, psoralens which find use in the present invention comprise a coumarin-like moiety fused with a furan ring. In some embodiments, the present invention provides furocoumarin compounds, and derivatives thereof. In some embodiments, the present invention provides furocoumarin-like compounds. In some embodiments, psoralens comprise a coumarin-like moiety, a furan ring, and one or more substituents and/or functional groups.

In some embodiments, the present invention provides substituted psoralen compounds (e.g. aminomethyl-4,5′,8-trimethylpsoralen (AMT); 8-methoxypsoralen (MOP); etc.) for use in enucleation of cells (e.g. feeder cells, eggs cells, sperm cells, etc.). In some embodiments, substituted psoralen compounds which find use in the present invention are substituted at one or more positions. In some embodiments, psoralen compounds which find use in the present invention are substituted by any chemical substituents understood by those of skill in the art, including, but not limited to cations, carboxylic acids, thicarboxylic acids, selenocarboxylic acids, sulfonic acids, sulfinic acids, sulfenic acids, esters, acyl halides, amides, imides, amidines, nitriles, aldehydes, ketones, alcohols, thiols, amines, imines, ethers, thioethers, peroxides, etc.

In some embodiments, the present invention provides one or more derivatives of psoralen (e.g. imperatorin, xanthotoxin, bergapten, nodekenetin, etc.). In some embodiments, the present invention provides a derivative of psoralen (e.g. imperatorin, xanthotoxin, bergapten, nodekenetin, etc.) comprising one or more substituent groups.

In some embodiments, psoralen (e.g. psoralen compound (e.g. AMT, MOP, etc.) is provided for cell enucleation in a concentration of 100 nm-1 mM (e.g. 100 nM . . . 200 nM . . . 500 nM . . . 1 μM . . . 2 μM . . . 5 μM . . . 10 μM . . . 20 μM . . . 50 μM . . . 100 μM . . . 200 μM . . . 500 μM . . . 1 mM). In some embodiments, psoralen (e.g. psoralen compound (e.g. AMT, MOP, etc.) is provided for cell enucleation in a concentration of 1-100 μM (e.g. about 50 μM). In some embodiments, psoralen compound concentration is dependent on the cell type undergoing enucleation. In some embodiments, the psoralen compound concentration is dependent on the duration psoralen compound of treatment. In some embodiments, psoralen compound concentration is dependent on the intensity of co-administered UV light. In some embodiments, psoralen compound concentration is dependent on the intensity of subsequently-administered UV light. In some embodiments, the psoralen compound concentration is dependent on the duration UV light treatment. In some embodiments, it is within the abilities of a skilled artisan to adjust the durations and concentrations of UV light and psoralen compound treatment to achieve effective cell enucleation.

In some embodiments, psoralen (e.g. psoralen compound (e.g. AMT, MOP, etc.) is provided for cell enucleation for a duration of 10 seconds to 10 hours (e.g. 10 seconds . . . 20 seconds . . . 1 minute . . . 2 minutes . . . 10 minutes . . . 20 minutes . . . 1 hour . . . 2 hours . . . 10 hours). In some embodiments, psoralen (e.g. psoralen compound (e.g. AMT, MOP, etc.) is provided for cell enucleation for a duration of 1-10 minutes (e.g. about 5 minutes). In some embodiments, psoralen compound treatment duration is dependent on the cell type undergoing enucleation. In some embodiments, the psoralen compound treatment duration is dependent on the concentration of psoralen compound of treatment. In some embodiments, psoralen compound treatment duration is dependent on the intensity of co-administered UV light. In some embodiments, psoralen compound treatment duration is dependent on the intensity of subsequently-administered UV light. In some embodiments, the psoralen compound treatment duration is dependent on the duration UV light treatment. In some embodiments, it is within the abilities of a skilled artisan to adjust the durations and concentrations of UV light and psoralen compound treatment to achieve effective cell enucleation.

In some embodiments, the present invention exposes cells (e.g. egg cells, feeder cells, sperm cells, etc.) to UV light. In some embodiments, the present invention exposes cells (e.g. egg cells, feeder cells, sperm cells, etc.) to UV light in the presence of one or more psoralen compounds. In some embodiments, the present invention exposes cells to 10 nm-400 nm wavelength light. In some embodiments, the present invention exposes cells to long-wave UV light (e.g. 300-400 nm). In some embodiments, cells are exposed to the entire spectrum of long-wave UV light (e.g. 300-400 nm). In some embodiments, cells are exposed to a portion of the long-wave spectrum of UV light (e.g. 300-320 nm, 310-350 nm, 370-400 nm, etc.).

In some embodiments, UV light treatment (e.g. long-wave UV light treatment) is provided to cells with enucleation for a duration of 10 seconds to 10 hours (e.g. 10 seconds . . . 20 seconds . . . 1 minute . . . 2 minutes . . . 10 minutes . . . 20 minutes . . . 1 hour . . . 2 hours . . . 10 hours). In some embodiments, UV light treatment (e.g. long-wave UV light treatment) is provided for 1-10 minutes (e.g. about 5 minutes).

In some embodiments, cells are incubated in one or more psoralen compounds and administered UV-light treatment concurrently. In some embodiments, cells are pre-treated with one or more psoralens for a duration (e.g. 1 minute . . . 2 minutes . . . 5 minutes . . . 10 minutes . . . 20 minutes . . . 1 hour, etc.) prior to exposure to UV light. In some embodiments, psoralen treatment and UV light treatment are initiated simultaneously.

In some embodiments, the present invention provides enucleation of cells. In some embodiments, the present invention enucleates cells for use as feeder cells.

### Experimental

The following section provides exemplary embodiments of the present invention, and should not be considered to be limiting of its scope with regard to alternative embodiments that are not explicitly described herein.

### Example 1

### Compositions and Methods

Xenopus. Pigmented and albino frogs, as well as CMV-GFP(+) female frogs were used in experiments conducted during development of embodiments of the present invention.

Antibodies. Anti-phospho S-345 Chk1 antibody (Cell Signaling Technology, #2341), and Oct4 antibody (Santa Cruz Biotechnology, #sc-9081) were used in experiments conducted during development of embodiments of the present invention.

Egg Enucleation. Freshly squeezed Xenopus eggs were incubated in a solution of 50 μM AMT in 1×MMR (100 mM NaCl, 2 mM KCl, 2 mM CaCl₂, 1 mM MgCl₂, 5 mM HEPES pH 7.4) for 5 minutes. During the incubation the eggs were rotated with forceps so that the white spot (indicating the position of the metaphase spindle) was facing upward. The eggs were then irradiated from above with a 100 W ultraviolet light source (Zeiss HBO 100 W/2) equipped with a D350/50X filter (Chroma). The lens of the light source was adjusted to give uniform UV intensity in the illuminated area, and the sample was placed 15.5 cm from the bulb. The beam intensity measured with a J221 long-wave UV meter (Ultraviolet Products, UVP) was approximately 15 mW/cm². The ultraviolet lamp was air-cooled with a small fan to prevent over-heating. Immediately after irradiation the eggs were fertilized and dejellied according to standard protocols (Murray, 1991) and allowed to develop in 0.1×MMR. Short-wave UV light was delivered with a UVGL-25 Mineralite (UVP) as described previously (Gurdon, 1960; Kroll and Gerhart, 1994).

Sperm Enucleation. A small piece of fresh Xenopus testis was macerated with forceps, resuspended in 1×MMR containing 1-50 μM AMT, then filtered through a 35 μM cell strainer (Falcon). The sperm suspension was spread onto a Petri dish then irradiated as described above and used immediately to fertilize fresh eggs. Mouse epididymal sperm were isolated in HTF medium (Quinn, 2000) and not filtered before irradiation.

Feeder Cell Enucleation. Cells were washed with Dulbecco's phosphate buffered saline (PBS) and incubated in PBS containing either 5 μM AMT or 5 μM MOP for 5 minutes. Cells were irradiated from above for 30-60 seconds with a 4 W UVGL-25 Mineralite (UVP) using the long-wave setting, holding the lamp as close as possible to the brim of the culture dish with the lid removed. After irradiation cells were washed with PBS. H7/WA07 hESCs (National Stem Cell Bank) were plated on primary MEFs 24 hours later. These MEFs were between passages 2 and 5. For keratinocytes precursor cell culture, the MEFS were trypsinized immediately after treatment and plated with CIN612 cells.

Karyotype Analysis. Karyotype analysis was performed using a modified protocol (http:// followed “by faculty.virginia.edu/xtropicalis/KaryotypeXtropicalis.htm”). Stage 38-36 tailbud embryos were incubated in 0.1×MMR containing 10 μM nocodazole for 1-3 hours. The tailbud was bluntly dissected off with sharp needles, incubated in 60% acetic acid for 5 minutes, and squashed onto a Superfrost Plus microscope slide (V WR Scientific) under a coverslip and a lead brick. After 5 minutes, the slide was quick-frozen on a cake of dry ice, the coverslip was prized off with a razor blade, and the squashed tissue was stained with 0.1 μg/ml 4′,6-diamidino-2-phenylindole (DAPI). At least three high quality spreads were counted before assigning a karyotype.

Primary Cell Culture. Fertilized eggs were cultured in sterile 0.1×MMR containing penicillin and streptomycin. The medium was changed daily and the embryos were transferred to fresh dishes after hatching. When embryos reached stage 40, a group of 20 embryos was homogenized by pipetting up and down with a sterile 9 inch Pasteur pipette in 2 ml culture medium (70% Liebovitz medium containing 10% fetal bovine serum, glutamine, and penicillin-streptomycin). The homogenate was diluted to 20 ml with culture medium and plated on an 89 mm tissue culture dish. After 48 hours, tissue fragments were aspirated off and the adherent cells were cultured for a week, split 1:2, and then cultured for another week. Cells were trypsinized, stained with propidium iodide, and analyzed by flow cytometry according to standard protocols (Darzynkiewicz and Juan, 2001).

Nuclear Transplantation. Blastulae were dissociated into single cells by incubation in Dissociation Medium (2.5 mM NaHCO₃, 7.5 mM Tris-HCl pH 7.6, 88 mM NaCl, 1 mM KCl, 0.5 mM EDTA) in an agarose-coated Petri dish. Recipient eggs were dejellied with 2% cysteine pH 7.8 then placed animal pole upwards in 1×MMR containing 25 μM MOP for 5 minutes then irradiated as described above for 5 minutes. Nuclear transfer was performed essentially as described by Gurdon (Gurdon, 1991).

### Example 2

### Psoralen Enucleation of Egg Cells

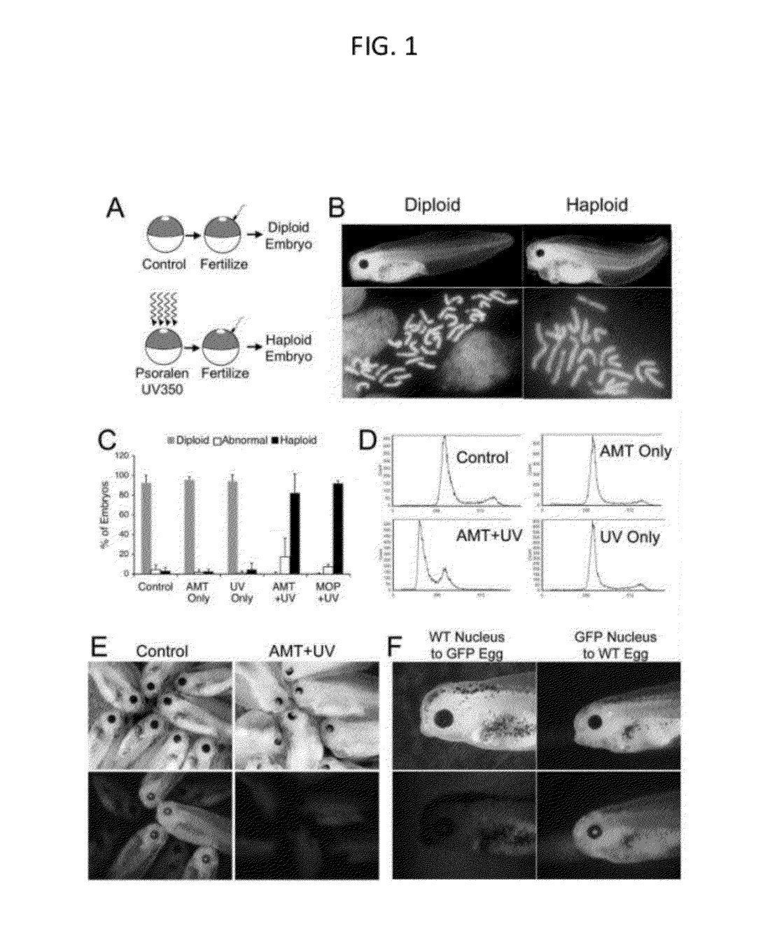

Experiments were conducted during development of embodiments of the present invention to determine whether psoralen treatment would prevent nuclear replication. Freshly laid Xenopus eggs were incubated in 50 μM 4′-aminomethyl -4, 5′, 8-trimethylpsoralen (AMT) for 5 minutes and manually rotated so that the white spot (indicating the position of the meiosis II spindle) was facing upward (SEE FIG. 1A). The eggs were irradiated from above for 5 minutes with a 100 W UV source outfitted with a 300-400 nm filter. After irradiation, the eggs were fertilized and allowed to develop in vitro. Both diploid and haploid Xenopus embryos develop to the swimming tadpole stage and can be distinguished by their physical appearance (Gurdon, 1960) (SEE FIG. 1B). Diploid embryos are elongated and tapered while haploid embryos are foreshortened, plump, and edematous. Eggs irradiated in the presence of AMT before fertilization gave rise to tadpoles with a typical haploid appearance (SEE FIG. 1C), indicating that the egg nucleus had not replicated. Karyotype analysis confirmed that embryos derived from AMT+UV-treated eggs had 18 chromosomes while embryos derived from untreated eggs had the normal diploid complement of 36 (Tymowska and Kobel, 1972) (SEE FIG. 1B and Table 1).

TABLE 1
Karyotype Analysis of Psoralen-treated and Control Embryos

| | Chromosome Number | |
|---|---|---|
| Treatment | (Mean ± SD) | # Embryos |
| Control | 36.0 ± 0.3 | 8 |
| AMT + UV | 18.0 ± 0.0 | 28 |
| MOP + UV | 18.0 ± 0.0 | 4 |
| Short Wave UV | 18.0 ± 0.0 | 3 |
| Abnormal | 35.4 ± 8.7* | 13 |
| Blastula SCNT | 37.2 ± 3.5 | 9 |
| Sperm AMT + UV | 18.0 ± 0.0 | 27 |

Primary cell cultures were established from pools of 20 tadpoles and measured the DNA content of individual cells by flow cytometry. Somatic cells derived from AMT+UV-treated eggs had a DNA content that was exactly half of that of cells derived from untreated eggs (SEE FIG. 1D). Eggs that were treated with AMT-only or UV-only developed into normal diploid embryos whose cells had the same DNA content as untreated controls.

Experiments were conducted during development of embodiments of the present invention to demonstrate that an AMT+UV-treated egg nucleus does not contribute genetically to embryonic development. A strain of transgenic frogs was employed that express GFP under the control of the ubiquitous CMV promoter (Marsh-Armstrong et al., 1999). When untreated CMV-GFP(+) eggs were fertilized with GFP(−) sperm, 63% of the embryos expressed GFP, indicating that the mother was heterozygous for the CMV-GFP allele (SEE FIG. 1E, left). In contrast, when the eggs of a CMV-GFP(+) female were treated with AMT and UV light before fertilization, virtually none of the haploid embryos expressed GFP (SEE FIG. 1E, right). The single exception was a mosaic that expressed GFP only in a patch of cells along the spine. Karyotype analysis confirmed that the GFP(−) tadpoles from AMT+UV-treated eggs had only 18 chromosomes. The karyotype results exclude the possibility that the embryos became GFP(−) due to psoralen-induced point mutations at the GFP locus.

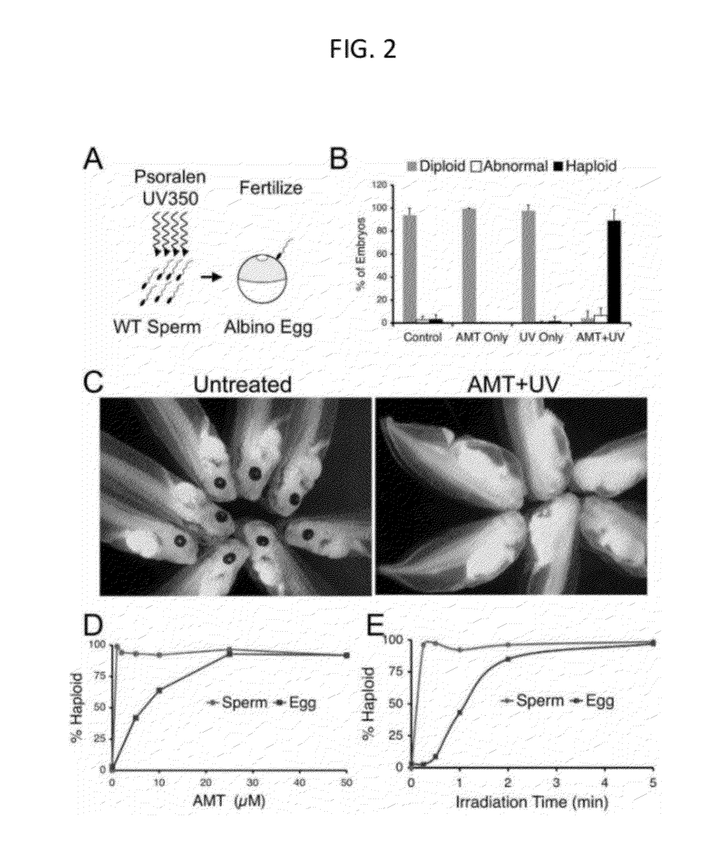

Enucleation of Xenopus eggs with psoralen and ultraviolet light is robust and reproducible. Among 20 different clutches of eggs from different mothers, the average efficiency of enucleation was 87±11%. The eggs were not damaged by psoralen+UV treatment, as evidenced by their high rates of fertilization and development into haploid tadpoles (80-100%). 8-methoxypsoralen (MOP) was substituted for AMT with little change in the efficiency of enucleation (SEE FIG. 1C). Irradiation of Xenopus eggs for 2-5 minutes in the presence of 25-50 μM AMT provided ideal enucleation conditions (SEE FIGS. 2D and 2E). Eggs irradiated for shorter periods of time or with lower concentrations of AMT produced abnormal embryos that either died during early development or gave rise to abnormally shaped tadpoles that resembled neither diploids nor haploids (not shown). Karyotype analysis showed that most surviving embryos had 32-34 chromosomes or were close to triploid (Table 1). Xenopus eggs treated by irradiation with short-wave UV light (Gurdon, 1960) were fertilized poorly compared to psoralen-treated eggs.

Eggs from a CMV-GFP female were enucleated with MOP and ultraviolet light and transplanted with blastula nuclei from control embryos to demonstrate that psoralen-enucleated eggs were suitable recipients for nuclear transplantation. From 216 primary transfers, 21 embryos were obtained, 10 of which had a normal morphology at stage 40. None of the 21 embryos exhibited GFP fluorescence, indicating that the recipient egg's nucleus had been destroyed by the enucleation procedure (SEE FIG. 1F, left). The converse experiment was also conducted, in which control eggs were enucleated with MOP and ultraviolet light and transplanted with cells from CMV-GFP blastulae. Of these transplant embryos, 35 of 36 (97%) were GFP(+), indicating that development was directed by the transplanted nucleus (SEE FIG. 1F, right). Karyotype analysis revealed that 8 of 9 of these GFP(+) transplant embryos had the normal complement of 36±1 chromosomes (Table 1). Thus, the frequency of complete enucleation appears to be about 90%.

### Example 3

### Psoralen Enucleation of Sperm Cells

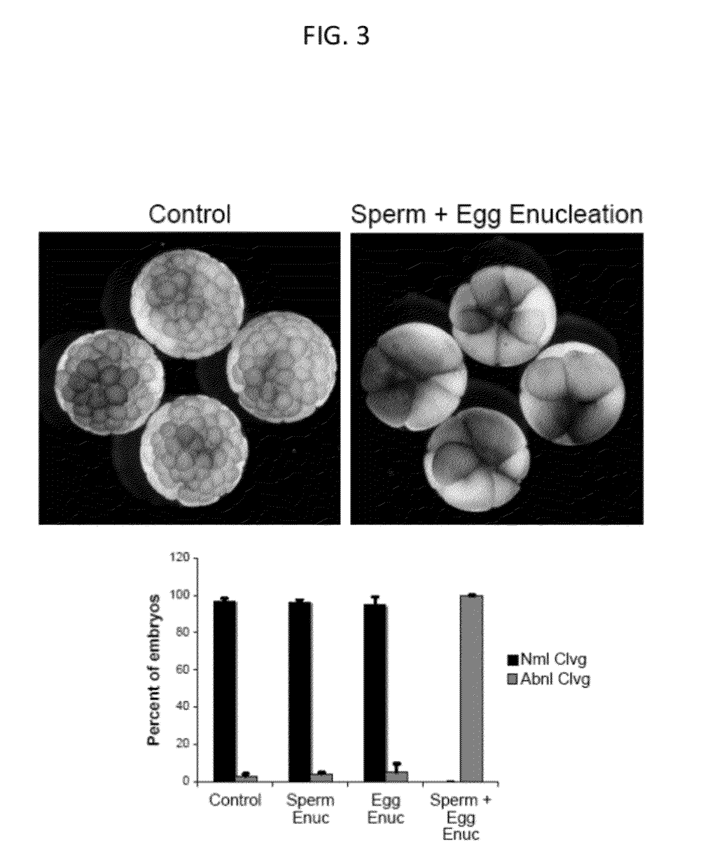

Experiments were conducted during development of embodiments of the present invention to demonstrate enucleation of other types of cells by psoralens. Sperm from a pigmented Xenopus male were treated with AMT and UV light then used to fertilize eggs from an albino female (SEE FIG. 2A). Virtually all of the tadpoles that developed had a typical haploid appearance and all were albino, indicating that the sperm nucleus did not contribute to the embryonic genome (SEE FIGS. 2, B and C). Karyotype analysis confirmed that the haploid-appearing embryos had 18 chromosomes (Table 1). Untreated sperm and sperm exposed to long-wave UV light without psoralen gave rise to normal diploid pigmented embryos (SEE FIG. 2B, left). Experiments demonstrated that the sperm nucleus is much more sensitive to psoralen and UV light than the egg nucleus. The efficiency of enucleation was higher (95±3%), and a psoralen concentration as low as 1 μM or an exposure to UV light as short as 15 seconds was sufficient to completely destroy the nucleus (SEE FIGS. 2E and 2F). The increased sensitivity is likely because the sperm nucleus is less shielded by cytoplasm, although the present invention is not limited to any particular mechanism of action and an understanding of the mechanism of action is not necessary to practice the present invention. Indeed, sperm that were treated with AMT but not exposed to UV light sometimes gave rise to abnormal embryos unless fertilization was performed in a darkroom under a safelight. Experiments indicated that ambient room light contains enough long-wave UV radiation to cause some degree of cross-linking. Treatment of sperm with AMT and UV light had no effect on the efficiency of fertilization. When both sperm and egg were treated with AMT+UV light, fertilization was normal and the eggs underwent several irregular cleavage divisions before dying during the blastula stage (SEE FIG. 3).

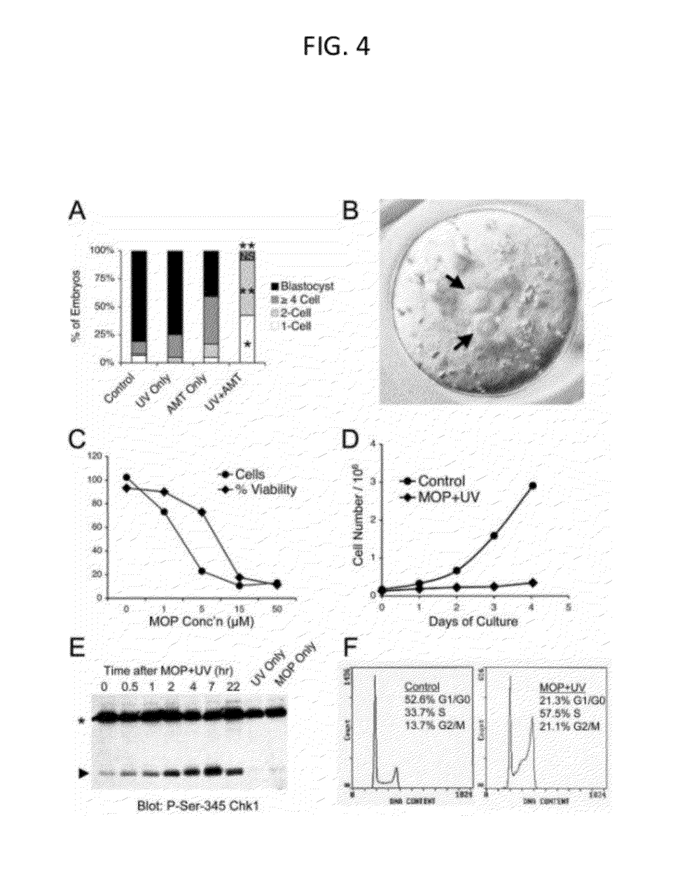

Experiments conducted during development of embodiments of the present invention also demonstrated that psoralens can effectively enucleate mouse eggs and sperm. Mouse eggs were much more sensitive to psoralens and ultraviolet light than Xenopus eggs, likely because they are smaller and more translucent, although the present invention is not limited to any particular mechanism of action and an understanding of the mechanism of action is not necessary to practice the present invention. Both the concentration of psoralen and the dose of UV light had to be reduced from the conditions used for frog eggs; otherwise, either individual treatment caused adverse effects on embryonic development in the absence of the other treatment. Eggs from super-ovulated mice were irradiated for 30 seconds with a handheld 4W UV lamp in the presence of 4-6 μM AMT, then parthenogenetically activated with strontium chloride and cultured in vitro for four days. AMT+UV-treated eggs arrested development at the 1-2 cell stage, while untreated eggs or eggs treated with UV light only or AMT only formed complete blastocysts (SEE FIG. 4A). Mouse sperm cells treated with AMT+UV light successfully fertilized mouse eggs in vitro, indicated by the presence of two pronuclei (SEE FIG. 3B). The fertilized zygotes, however, rarely cleaved even once, while 20-40% of eggs fertilized with untreated sperm developed to the blastocyst stage. These results indicate that, unlike Xenopus eggs, mouse eggs fertilized with AMT+UV-treated sperm arrest development at the first cell cycle. The arrest is likely due to activation of cell cycle checkpoints by the psoralen-damaged DNA, although the present invention is not limited to any particular mechanism of action and an understanding of the mechanism of action is not necessary to practice the present invention. In Xenopus embryos checkpoints do not become operational until the 12th cell division (Gerhart et al., 1984), by which time the psoralen-damaged DNA would have been segregated to a small group of cells. In mouse embryos, however, checkpoints are active during the early cleavage divisions (Liu et al., 2000b; Takai et al., 2000).

### Example 4

### Production of Feeder Cells with Psoralens

Experiments were conducted during development of embodiments, of the present invention to demonstrate the use of psoralen-treated cells as feeder cells. The effects of psoralen+UV treatment on the growth of HeLa cells were examined. Cells were treated with various concentrations of MOP, irradiated with long wave UV light for 1 minute, and then cultured for three days. A dose of 5 μM MOP effectively arrested HeLa cell growth while maintaining cell viability at 80-100% (SEE FIG. 4C). Flow cytometry of the MOP+UV-treated cells demonstrated that they were arrested in S and G2/M phases of the cell cycle, indicating that DNA replication was blocked (SEE FIG. 4F). The arrested cells had high levels of S345-phosphorylated Chk1, indicating activation of the DNA replication checkpoint pathway (SEE FIG. 4E). Similar results have been reported previously (Pichierri and Rosselli, 2004). When grown in culture, MOP+UV-treated HeLa cells did not proliferate while untreated cells grew with a doubling time close to 24 hours (SEE FIG. 4D). Cells exposed to long-wave UV light in the absence of MOP or cells incubated in MOP without UV exposure grew normally and showed little or no Chk1 phosphorylation.

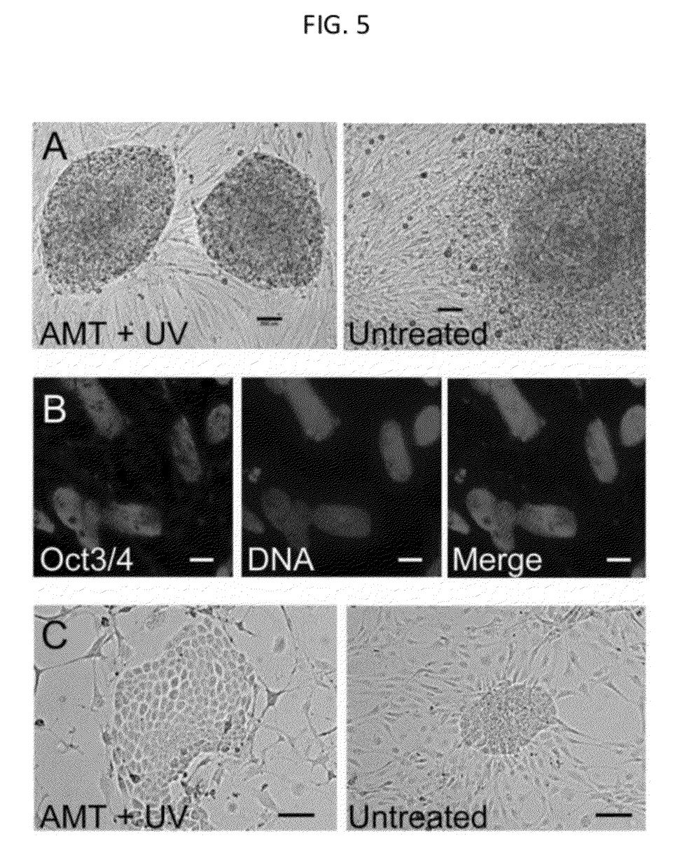

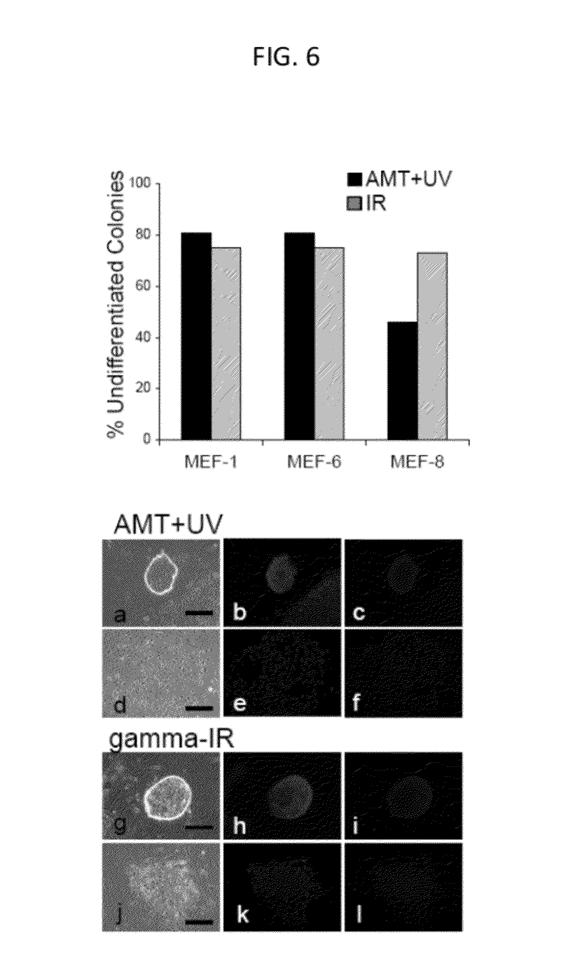

Human embryonic stem cells (hESCs) require a feeder layer of mouse embryonic fibroblasts (MEFs) to grow in culture (Thomson et al., 1998). To determine whether psoralen-treated MEFs could be used as feeders, H7/WA07 hESCs were plated on a monolayer of primary MEFs that had been treated with 5 μM AMT and irradiated with long-wave ultraviolet light for 30 seconds. After 5 days, healthy-appearing hESC colonies with smooth rounded borders were growing on the treated MEFs (SEE FIG. 5A). Colonies were indistinguishable from those grown on gamma-irradiated MEFs. The hESCs expressed normal amounts of the ES cell-specific transcription factor Oct3/4 (SEE FIG. 5B), indicating that they maintained their undifferentiated state. When plated on untreated MEFs, hESC colonies had indistinct borders suggesting the cells had differentiated prematurely. The ability of MEFs to support hESC growth declines with time (VIIIa-Diaz et al., 2009). AMT+UV-treated MEFs would maintain hESCs in an undifferentiated state when plating was done within 6 days of treatment, but that after 8 days there was a noticeable decrease in the proportion of undifferentiated colonies (SEE FIG. 6).

The human keratinocyte precursor cell line CIN612 is widely used in studies of human papillomavirus (HPV) (Ozbun, 2002). CIN612 cells harbor episomal copies of HPV31b, a subtype that causes cervical cancer. The cells are typically grown on a feeder layer of mouse fibroblasts. Mouse J2 3T3 fibroblasts were treated with 5 μM AMT and UV light for 2 minutes, then trypsinized and plated with CIN612 cells. After 3-7 days in culture, small colonies of CIN612 cells appeared that were indistinguishable from those grown on mitomycin C-treated feeder cells (SEE FIG. 5C, left). When untreated cells were used as feeders, the colonies were much smaller (SEE FIG. 5C, right). The growth rate of CIN612 cells on psoralen-treated feeders was the same as on mitomycin-C treated feeders.

The references cited above, and/or listed below are herein incorporated by reference in their entireties. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

### REFERENCES

1. Cimino G D, Gamper H B, Isaacs S T, Hearst J E. Psoralens as photoactive probes of nucleic acid structure and function: organic chemistry, photochemistry, and biochemistry. Annu Rev Biochem. 1985; 54:1151-1193.
2. Cowan C A, Atienza J, Melton D A, Eggan K. Nuclear reprogramming of somatic cells after fusion with human embryonic stem cells. Science. 2005; 309:1369-1373.
3. Darzynkiewicz Z, Juan G. DNA Content Measurement for DNA Ploidy and Cell Cycle Analysis. In: Robinson J, editor. Current Protocols in Cytometry. New York, N.Y.: Wiley; 2001.
4. Fulka J, Jr, Moor R M. Noninvasive chemical enucleation of mouse oocytes. Mol Reprod Dev. 1993; 34:427-430.
5. Gerhart J, Wu M, Kirschner M. Cell cycle dynamics of an M-phase-specific cytoplasmic factor in Xenopus laevis oocytes and eggs. J. Cell Biol. 1984; 98:1247-1255.
6. Gurdon J. The Effects of Ultraviolet Radiation on Uncleaved Eggs of Xenopus laevis. Quarterly Journal of Microscopical Science. 1960; 101:299-311.
7. Gurdon J B. Nuclear transplantation in Xenopus. Methods Cell Biol. 1991; 36:299-309.
8. Kane D A, Kimmel C B. The zebrafish midblastula transition. Development. 1993; 119:447-456.
9. Kawakami M, Tani T, Yabuuchi A, Kobayashi T, Murakami H, Fujimura T, Kato Y, Tsunoda Y. Effect of demecolcine and nocodazole on the efficiency of chemically assisted removal of chromosomes and the developmental potential of nuclear transferred porcine oocytes. Cloning Stem Cells. 2003; 5:379-387.
10. Kroll K L, Gerhart J C. Transgenic X. laevis embryos from eggs transplanted with nuclei of transfected cultured cells. Science. 1994; 266:650-653.
11. Li G P, White K L, Bunch T D. Review of enucleation methods and procedures used in animal cloning: state of the art. Cloning Stem Cells. 2004; 6:5-13.
12. Li J, Du Y, Zhang Y H, Kragh P M, Purup S, Bolund L, Yang H, Xue Q Z, Vajta G. Chemically assisted handmade enucleation of porcine oocytes. Cloning Stem Cells. 2006; 8:241-250.
13. Liu L, Oldenbourg R, Trimarchi J R, Keefe D L. A reliable, noninvasive technique for spindle imaging and enucleation of mammalian oocytes. Nat. Biotechnol. 2000a; 18:223-225.
14. Liu Q, Guntuku S, Cui X S, Matsuoka S, Cortez D, Tamai K, Luo G, Carattini-Rivera S, DeMayo F, Bradley A, Donehower L A, Elledge S J. Chk1 is an essential kinase that is regulated by Atr and required for the G(2)/M DNA damage checkpoint. Genes & Development. 2000b; 14:1448-1459.
15. Marsh-Armstrong N, Huang H, Berry D L, Brown D D. Germ-line transmission of transgenes in Xenopus laevis. Proc Natl Acad Sci USA. 1999; 96:14389-14393.
16. Murray A W. Cell cycle extracts. Methods Cell Biol. 1991; 36:581-605.
17. O'Farrell P H, Stumpff J, Su T T. Embryonic cleavage cycles: how is a mouse like a fly? Curr Biol. 2004; 14:R35-45.
18. Ozbun M A. Infectious human papillomavirus type 31b: purification and infection of an immortalized human keratinocyte cell line. J Gen Virol. 2002; 83:2753-2763.
19. Pichierri P, Rosselli F. The DNA crosslink-induced S-phase checkpoint depends on ATR-CHK1 and ATR-NBS1-FANCD2 pathways. Embo J. 2004; 23:1178-1187.
20. Quinn P. Review of media used in ART laboratories. J. Androl. 2000; 21:610-615.
21. Raff J W, Glover D M. Nuclear and cytoplasmic mitotic cycles continue in Drosophila embryos in which DNA synthesis is inhibited with aphidicolin. J. Cell Biol. 1988; 107:2009-2019.
22. Smith L C. Membrane and intracellular effects of ultraviolet irradiation with Hoechst 33342 on bovine secondary oocytes matured in vitro. J Reprod Fertil. 1993; 99:39-44.
23. Stern R S. Psoralen and ultraviolet a light therapy for psoriasis. N Engl J. Med. 2007; 357:682-690.
24. Takai H, Tominaga K, Motoyama N, Minamishima Y A, Nagahama H, Tsukiyama T, Ikeda K, Nakayama K, Nakanishi M, Nakayama K. Aberrant cell cycle checkpoint function and early embryonic death in Chk1(−/−) mice. Genes & Development. 2000; 14:1439-1447.
25. Tatham B G, Dowsing A T, Trounson A O. Enucleation by centrifugation of in vitro-matured bovine oocytes for use in nuclear transfer. Biol Reprod. 1995; 53:1088-1094.
26. Thomson J A, Itskovitz-Eldor J, Shapiro S S, Waknitz M A, Swiergiel J J, Marshall V S, Jones J M. Embryonic stem cell lines derived from human blastocysts. Science. 1998; 282:1145-1147.
27. Tymowska J, Kobel H R. Karyotype analysis of Xenopus muelleri (Peters) and Xenopus laevis (Daudin), Pipidae. Cytogenetics. 1972; 11:270-278.
28. Vajta G, Maddox-Hyttel P, Skou C T, Tecirlioglu R T, Peura T T, Lai L, Murphy C N, Prather R S, Kragh P M, Callesen H. Highly efficient and reliable chemically assisted enucleation method for handmade cloning in cattle. Reprod Fertil Dev. 2005; 17:791-797.
29. VIIIa-Diaz L G, Pacut C, Slawny N A, Ding J, O'Shea K S, Smith G D. Analysis of the factors that limit the ability of feeder cells to maintain the undifferentiated state of human embryonic stem cells. Stem Cells Dev. 2009; 18:641-651.
30. Wang M K, Liu J L, Li G P, Lian L, Chen D Y. Sucrose pretreatment for enucleation: an efficient and non-damage method for removing the spindle of the mouse MII oocyte. Mol Reprod Dev. 2001; 58:432-436.

We claim:

1. A method of enucleating cells comprising treating cells with a psoralen compound and UV light.

2. The method of claim 1, wherein said psoralen compound comprises a substituted psoralen compound.

3. The method of claim 2, wherein said psoralen compound comprises aminomethyl-4,5′,8-trimethylpsoralen (AMT).

4. The method of claim 2, wherein said psoralen compound comprises 8-methoxypsoralen (MOP).

5. The method of claim 1, wherein said psoralen compound comprises an isomer or derivative of psoralen.

6. The method of claim 1, wherein said cells are incubated with said psoralen compound for 1-10 minutes.

7. The method of claim 1, wherein said cells are incubated with said psoralen compound for 1-10 minutes prior to exposure with UV light.

8. The method of claim 1, wherein said UV light comprises long-wave UV light.

9. The method of claim 1, wherein said psoralen compound and said UV light are administered concurrently.

10. The method of claim 1, wherein said cells are exposed to said UV light for 1-10 minutes.

11. The method of claim 1, wherein said UV light is administered subsequent to said psoralen compound.

12. The method of claim 1, wherein said cells comprise eggs cells or sperm cells.

13. A composition comprising one or more enucleated cells, wherein said cells have been enucleated by exposure to a psoralen compound and UV light.

14. The composition of claim 13, wherein said cells comprise eggs cells or sperm cells.

15. The composition of claim 13, wherein said cells comprise feeder cells.

16. The composition of claim 13, wherein said psoralen compound comprises AMT or MOP.

17. The composition of claim 13, wherein said UV light comprises long-wave UV light.

18. The composition of claim 13, wherein said cells comprise a nucleus.

19. The composition of claim 18, wherein said cells are incapable of DNA replication.

20. The composition of claim 19, wherein the DNA of said cells comprise covalent cross-strand cross-links.

21. A system comprising:
a) a device configured to produce UV light;
b) a psoralen compound; and
c) a plurality of cells.

22. The system of claim 21, wherein said cells are eggs cells.

23. The system of claim 21, wherein said cells are sperm cells.
